(19) United States
(12) Patent Application Publication
(10) Pub. No.: US 2025/0302746 A1
(43) Pub. Date: Oct. 2, 2025

(54) METHOD FOR PRODUCING EMULSIFIED COMPOSITION

(71) Applicant: Daicel Corporation, Osaka-shi, Osaka (JP)

(72) Inventors: Takanori SOMEYA, Myoko-shi, Niigata (JP); Shoichi SAWADA, Tokyo (JP); Kenichi OOE, Tokyo (JP)

(73) Assignee: Daicel Corporation, Osaka-shi, Osaka (JP)

(21) Appl. No.: 18/855,905

(22) PCT Filed: Apr. 11, 2023
(86) PCT No.: PCT/JP2023/014667

§ 371 Date: Oct. 10, 2024

(30) Foreign Application Priority Data
Apr. 11, 2022 (JP) 2022-064910

(51) Int. Cl. A61K 9/107; A61K 31/37

(52) U.S. Cl. CPC A61K 9/107; A61K 31/37

(57) ABSTRACT

The present disclosure provides a technique for producing a composition in which at least urolithins are solubilized, and a method for producing an emulsified composition.

### TECHNICAL FIELD

The present disclosure relates to a method for producing an emulsified composition.

### BACKGROUND ART

Among various physiologically active substances utilized as materials for food and beverage products, pharmaceutical products, cosmetic products, and the like, there are many substances that are poorly soluble. When such a poorly soluble substance can be blended in the form of powder or crystal, the poorly soluble substance may be used as it is, but when the substance is used as it is in the case where it is desired to blend the poorly soluble substance in a solubilized state, aggregation or precipitation occurs.

When a poorly soluble substance is solubilized, a method called emulsification may be employed.

For example, it has been reported that in order to blend a poorly water-soluble component such as curcuminoid into a processed food containing water, the average emulsified particle diameter of a water-diluted solution thereof is set to a predetermined range by blending an emulsifier and a polyhydric alcohol (Patent Document 1).

It has also been reported that when a polyphenol is emulsified in the presence of an emulsifier and an oily component, micronization of the resulting emulsified particles is promoted, formation of coarse particles is suppressed, and emulsion stability is improved (Patent Document 2).

It has also been reported that when a poorly water-soluble component such as resveratrol is added to a food oil of 100° C. or higher, and the component is dissolved, emulsified and dispersed, a poorly water-soluble component-containing drug product in which the poorly water-soluble component is stably dispersed can be produced (Patent Document 3).

Known examples of the physiologically active substances used as materials for food and beverage products, pharmaceutical products, cosmetic products, and the like include urolithins such as urolithin A and urolithin C. For example, urolithin A has been reported to have functions such as an antioxidant effect (Non-Patent Literature 1), an anti-inflammatory effect (Non-Patent Literature 2), an anti-glycation effect (Non-Patent Literature 3), and a mitophagy-promoting effect (Non-Patent Literature 4).

Urolithins are also poorly soluble substances, and for example, it is known that the solubility of urolithin A in water at 30° C. is about 3.5 mg/L (Patent Document 4). In addition, it has low oil solubility and is not soluble in high-temperature vegetable oil or the like. Thus, a composition in which urolithins are solubilized cannot be produced by the above-described solubilization method.

On the other hand, a method for solubilizing urolithins using cyclodextrin has been reported (Patent Document 4). However, even when this method is used, it is necessary to contain a large amount of cyclodextrin in order to increase the concentration of the solubilized urolithins, and there is room for improvement.

### PRIOR ART DOCUMENTS

### Patent Document

Patent Document 1: JP 2021-052742 A
Patent Document 2: JP 2021-016380 A
Patent Document 3: JP 2018-43945 A
Patent Document 4: JP 2017-218434 A

### Non-Patent Literature

Non-Patent Literature 1: Biosci. Biotechnol. Biochem., 76, 395-399 (2012)
Non-Patent Literature 2: J. Agric. Food Chem., 60, 8866-8876 (2012)
Non-Patent Literature 3: Mol. Nutr. Food Res., 55, S35-S43 (2011)
Non-Patent Literature 4: Nature Medicine, 22, 879-888 (2016)

### SUMMARY OF THE INVENTION

### Problems to be Solved by the Invention

An object of the present disclosure is to provide a technique for producing a composition in which at least urolithins are solubilized.

### Means for Solving the Problems

The inventors of the present invention have found that an emulsified composition in which urolithins are solubilized can be produced by heating a solution under predetermined conditions containing urolithins, an emulsifier, and a polyhydric alcohol under predetermined conditions.

In one aspect of the present disclosure, a method for producing an emulsified composition includes steps (a) or (b), wherein the emulsified composition has a transmittance of 40% or greater for light having a wavelength of 660 nm at a measurement optical path length of 10 mm:
(a)
   heating a solution containing urolithins, an emulsifier, and a polyhydric alcohol at 120° C. or higher for 1 minute or more, wherein the emulsifier has an HLB of 12.0 or greater, and a total amount of the emulsifier is 50 parts by weight or more with respect to 1 part by weight of the urolithins in total;
(b)
   heating a solution containing urolithins, an emulsifier, and a polyhydric alcohol at 105° C. or higher for 1 minute or more, wherein the emulsifier has an HLB of 15.5 or greater, and a total amount of the emulsifier is 500 parts by weight or more with respect to 1 part by weight of the urolithins in total.

In a preferred aspect of the production method, the polyhydric alcohol is glycerol.

In a preferred aspect of the production method, the urolithins are urolithin A, urolithin B, urolithin C, urolithin M5, urolithin M6, urolithin M7, urolithin M8, or isourolithin A.

In another aspect of the present disclosure, an emulsified composition contains urolithins, an emulsifier, and a polyhydric alcohol, wherein the emulsifier has an HLB of 12.0 or greater, a total amount of the emulsifier is 50 parts by weight or more with respect to 1 part by weight of the urolithins in total, and the emulsified composition has a transmittance of 40% or greater for light having a wave length of 660 nm at a measurement optical path length of 10 mm.

In a preferred aspect of the emulsified composition, the polyhydric alcohol is glycerol.

In a preferred aspect of the emulsified composition, the urolithins are urolithin A, urolithin B, urolithin C, urolithin M5, urolithin M6, urolithin M7, urolithin M8, or isourolithin A.

In another aspect of the present disclosure, a product contains the emulsified composition.

In a preferred aspect, the product is a food or beverage product, a pharmaceutical product, or a cosmetic product.

### Effect of the Invention

The present disclosure can provide an effect of providing a technique for producing a composition in which at least urolithins are solubilized. Thus, according to the present technique, for example, urolithins, which are poorly soluble substances, can be solubilized, and thus the urolithins can be blended in a solubilized state into a liquid composition.

The present disclosure can also provide an effect that when the composition is administered to a subject, the bioavailability of the urolithins in the subject is significantly larger than when the urolithins are administered in the form of a crystalline powder.

### MODE FOR CARRYING OUT THE INVENTION

Each of the configurations, combinations thereof, and the like in each of the embodiments are an example, and various additions, omissions, substitutions, and other changes of the configurations may be made as appropriate without departing from the spirit of the present disclosure. The present disclosure is not limited by the embodiments and is limited only by the claims.

In one embodiment of the present disclosure, a method for producing an emulsified composition includes steps (a) or (b) shown below, wherein the emulsified composition has a transmittance of 40% or greater for light having a wavelength of 660 nm at a measurement optical path length of 10 mm:
(a)
   heating a solution containing urolithins, an emulsifier, and a polyhydric alcohol at 120° C. or higher for 1 minute or more, wherein the emulsifier has an HLB of 12.0 or greater, and a total amount of the emulsifier is 50 parts by weight or more with respect to 1 part by weight of the urolithins in total;
(b)
   heating a solution containing urolithins, an emulsifier, and a polyhydric alcohol at 105° C. or higher for 1 minute or more, wherein the emulsifier has an HLB of 15.5 or greater, and a total amount of the emulsifier is 500 parts by weight or more with respect to 1 part by weight of the urolithins in total.

The emulsified composition produced by the production method according to the present embodiment has a transmittance of 40% or greater for light having a wavelength of 660 nm at a measurement optical path length of 10 mm. The transmittance for light having a wavelength of 660 nm at a measurement optical path length of 10 mm can be measured using, for example, an ultraviolet-visible spectrophotometer UV-1800 (Shimadzu Corporation).

In the present disclosure, it can be said that the urolithins are solubilized in the emulsified composition when the transmittance is 40% or greater. As described below, the emulsified composition is subjected to a heating step, then the temperature is lowered to room temperature (about 25° C.), and then the transmittance is measured. That is, the transmittance in the present disclosure is measured at room temperature (about 25° C.).

The transmittance is preferably, in ascending order of preference, 45% or greater, 50% or greater, 55% or greater, 60% or greater, 65% or greater, 70% or greater, 75% or greater, 80% or greater, 85% or greater, 90% or greater, or 95% or greater since it is preferable that the urolithins be more solubilized in the emulsified composition. On the other hand, the upper limit is not particularly limited, but is preferably larger, and is, for example, 100% or less, 99% or less, or the like. The upper limit and the lower limit may also be a consistent combination thereof. For example, the transmittance is from 40% to 100%, from 45% to 100%, from 50% to 100%, from 55% to 100%, from 60% to 100%, from 65% to 100%, from 70% to 99%, from 75% to 99%, from 80% to 99%, from 85% to 99%, from 90% to 99%, from 95% to 99%, or the like.

Next, the step (a) will be described.

The step (a) of the production method according to the present embodiment is a step of heating a solution containing urolithins, an emulsifier, and a polyhydric alcohol at 120° C. or higher for 1 minute or more, wherein the emulsifier has an HLB of 12.0 or greater, and
a total amount of the emulsifier is 50 parts by weight or more with respect to 1 part by weight of the urolithins in total.

The urolithins are represented by General Formula (1) shown below.

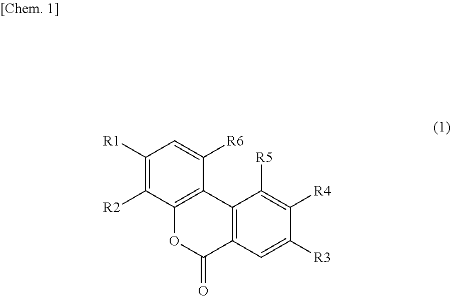

In the formula, R1 to R6 each independently represent a hydroxyl group, a hydrogen atom, or a methoxy group.

One or more of R1 to R6 in the formula may be hydroxyl groups, or all of them may be hydrogen atoms.

Examples of the urolithins include all the compounds represented by General Formula (1). Specific examples thereof include urolithin A, urolithin B, urolithin C, urolithin D, urolithin E, urolithin M3, urolithin M4, urolithin M5, urolithin M6, urolithin M7, urolithin M8, isourolithin A, and 6H-dibenzo[b,d]pyran-6-one.

Preferred is urolithin A, urolithin B, urolithin C, urolithin M5, urolithin M6, urolithin M7, urolithin M8, or isourolithin A.

In this step, a single type of the urolithins may be used alone or two or more types thereof in any combination.

In the present disclosure, there is a case where description is made using plural forms such as “urolithins”, but this is merely a formal notation in terms of classification. For example, the term “urolithins” is a concept including urolithin A, urolithin B, and the like categorized into “urolithins”, and “urolithins” may be simply referred to as “urolithin” as a term representing a superordinate concept of urolithin A, urolithin B, and the like.

In the solution containing the urolithins, the emulsifier, and the polyhydric alcohol, the total amount of the urolithins is, for example, 0.001 wt. % or greater, 0.005 wt. % or greater, 0.01 wt. % or greater, 0.05 wt. % or greater, 0.1 wt. % or greater, 0.5 wt. % or greater, or 1.0 wt. % or greater. The total amount of the urolithins is, for example, 10 wt. % or less, 5 wt. % or less, 2.5 wt. % or less, or 2 wt. % or less. The upper limit and the lower limit may also be a consistent combination thereof. For example, the total amount of the urolithins is from 0.001 wt. % to 10 wt. %, from 0.005 wt. % to 5 wt. %, from 0.01 wt. % to 2.5 wt. %, from 0.05 wt. % to 2 wt. %, from 0.1 wt. % to 2 wt. %, from 0.5 wt. % to 2 wt. %, from 1.0 wt. % to 2 wt. %, or the like.

As the emulsifier, one having a hydrophilic-lipophilic balance (HLB) of 12.0 or greater may be used.

In the present disclosure, the value of HLB is a value calculated from Griffin's equation, and the geometric proportion (critical packing parameter (CPP)) between the hydrophilic moiety and the hydrophobic moiety of the amphiphile is preferably from 1/2 to 1.

In this step, a single type of the emulsifier may be used alone or two or more types thereof in any combination.

It is preferable that the urolithins be more solubilized in the emulsified composition, and from this viewpoint, it is preferable that the HLB be larger. Thus, the HLB is, in ascending order of preference, 12.5 or greater, 12.9 or greater, 13.0 or greater, 13.4 or greater, 13.5 or greater, 14.0 or greater, 14.5 or greater, 14.7 or greater, 14.9 or greater, 15.0 or greater, 15.5 or greater, 15.7 or greater, 16.0 or greater, 16.1 or greater, 16.5 or greater, 16.7 or greater, 16.9 or greater, 17.0 or greater, 17.5 or greater, 18.0 or greater, 18.5 or greater, 19.0 or greater, or 19.5 or greater. The upper limit of the HLB is, for example, 20.0 or less. The upper limit and the lower limit may also be a consistent combination thereof. For example, the HLB is from 12.0 to 20.0, from 12.5 to 20.0, from 12.9 to 20.0, from 13.0 to 20.0, from 13.4 to 20.0, from 13.5 to 20.0, from 14.0 to 20.0, from 14.5 to 20.0, from 14.7 to 20.0, from 14.9 to 20.0, from 15.0 to 20.0, from 15.5 to 20.0, from 15.7 to 20.0, from 16.0 to 20.0, from 16.1 to 20.0, from 16.5 to 20.0, from 16.7 to 20.0, from 16.9 to 20.0, from 17.0 to 20.0, from 17.5 to 20.0, from 18.0 to 20.0, from 18.5 to 20.0, from 19.0 to 20.0, from 19.5 to 20.0, or the like.

Examples of the emulsifier include emulsifiers having an HLB within any of the above-described HLB ranges (for example, polyglycerol fatty acid ester, sucrose fatty acid ester, organic acid monoglyceride, propylene glycol fatty acid ester, polyglycerol condensed ricinoleic acid ester, sorbitan fatty acid ester, polyoxyethylene sorbitan fatty acid ester, and the like).

Examples of the polyglycerol fatty acid ester include those having an average degree of polymerization of glycerol of, for example, 4 or greater, 6 or greater, and on the other hand, 10 or less. That is, for example, one having an average degree of polymerization of glycerol of from 4 to 10, from 6 to 10, or the like. Examples of the number of carbons of the constituent fatty acid include 12 or more, and 18 or less. That is, for example, one having from 12 to 18 carbons. The constituent fatty acid may be a saturated or unsaturated fatty acid. Examples of the constituent fatty acid include caprylic acid, lauric acid, myristic acid, pentadecylic acid, palmitic acid, palmitoleic acid, margaric acid, stearic acid, and oleic acid.

Examples of the polyglycerol fatty acid ester include hexaglycerol monooleate, hexaglycerol monostearate, hexaglycerol monopalmitate, hexaglycerol monomyristate, hexaglycerol monolaurate, decaglycerol monooleate, decaglycerol monostearate, decaglycerol monopalmitate, decaglycerol monomyristate, and decaglycerol monolaurate.

Examples of the sucrose fatty acid ester include those in which the constituent fatty acid has 12 or more and 18 or less carbons. That is, for example, one having from 12 to 18 carbons. The constituent fatty acid may be a saturated or unsaturated fatty acid. Examples of the constituent fatty acid include lauric acid, myristic acid, pentadecylic acid, palmitic acid, palmitoleic acid, margaric acid, stearic acid, and oleic acid.

Examples of the sucrose fatty acid ester include sucrose dioleate, sucrose distearate, sucrose dipalmitate, sucrose dimyristate, sucrose dilaurate, sucrose monooleate, sucrose monostearate, sucrose monopalmitate, sucrose monomyristate, and sucrose monolaurate.

Examples of the sorbitan fatty acid ester include those in which the fatty acid has, for example, 8 or more carbons, or 12 or more carbons.

Examples of the sorbitan fatty acid ester include sorbitan monocaprylate, sorbitan monolaurate, sorbitan monostearate, sorbitan sesquistearate, sorbitan tristearate, sorbitan isostearate, sorbitan sesquiisostearate, sorbitan oleate, sorbitan sesquioleate, and sorbitan trioleate.

Examples of the polyoxyethylene sorbitan fatty acid ester include those in which the fatty acid has, for example, 8 or more carbons, or 12 or more carbons. The length (number of moles added) of ethylene oxide in polyoxyethylene is, for example, 2 or greater, or 4 or greater, and on the other hand, for example, 100 or less, or 50 or less. Examples thereof include one having the length of from 2 to 100, or from 4 to 50.

Examples of the polyoxyethylene sorbitan fatty acid ester include polyoxyethylene sorbitan monocaprylate, polyoxyethylene sorbitan monolaurate, polyoxyethylene sorbitan monostearate, polyoxyethylene sorbitan sesquistearate, polyoxyethylene sorbitan tristearate, polyoxyethylene sorbitan isostearate, polyoxyethylene sorbitan sesquiisostearate, polyoxyethylene sorbitan oleate, polyoxyethylene sorbitan sesquioleate, and polyoxyethylene sorbitan trioleate.

As the emulsifier, those widely used in the fields of food and beverage products, pharmaceutical products, and cosmetic products are preferable. An appropriate emulsifier can be selected depending on the final product of the emulsified composition produced by the production method according to the present embodiment (for example, a food or beverage product, a pharmaceutical product, or a cosmetic product).

An emulsifier is exemplified for each value of HLB. Product names starting with “NIKKOL” are commercially available products manufactured by Nikko Chemicals Co., Ltd., product names starting with “RYOTO (trade name)” are commercially available products manufactured by Mitsubishi Chemical Corporation, product names starting with “SY Glyster” are commercially available products manufactured by Sakamoto Yakuhin Kogyo Co., Ltd., and the product name starting with “EMASOL” is a commercially available product manufactured by Kao Corporation, which are listed as examples.

Examples of the emulsifier having an HLB of 12.0 include decaglyceryl monooleate (NIKKOL Decaglyn 1-OVF, NIKKOL Decaglyn 1-OV, NIKKOL Decaglyn 1-OVEXF), decaglyceryl monostearate (NIKKOL Decaglyn 1-SV, NIKKOL Decaglyn 1-SVF), decaglyceryl monoisostearate (NIKKOL Decaglyn 1-ISV), POE lanolin (NIKKOL TW-10), POE (7) secondary alkyl ether (NIKKOL BT-7), and POE (30) POP (6) decyl tetradecyl ether (NIKKOL SG-DTD630).

Examples of the emulsifier having an HLB of 12.5 include tetraoleic acid POE (40) sorbitol (NIKKOL GO-440V), POE (40) hydrogenated castor oil (NIKKOL HCO-40, NIKKOL HCO-40 (for pharmaceutical purpose)), POE (20) POP (8) cetyl ether (NIKKOL PBC-44), diPOE (8) sodium oleyl ether phosphate (NIKKOL DOP-8NV), polyethylene glycol monolaurate (10E.0.) (NIKKOL MYL-10), polyglycerol fatty acid ester (cosmetic component display name: polyglyceryl palmitate-10, quasi-drug component display name: glycerol fatty acid ester (*to be added to food)) (NIKKOL Decaglyn 1-PVEX), polyglyceryl stearate-10 (NIKKOL Decaglyn 1-SVEX), polyoxyethylene phytosterol (NIKKOL BPS-10), decaglyceryl monopalmitate (NIKKOL Decaglyn 1-PVEXF), and decaglyceryl monostearate (NIKKOL Decaglyn 1-SVEXF).

Examples of the emulsifier having an HLB of 12.9 include polyglycerol monooleate (SY Glyster MO-7S).

Examples of the emulsifier having an HLB of 13.0 include decaglycerol oleate (RYOTO (trade name) polyglyester O-15D), tetrastearic acid POE (60) sorbitol (NIKKOL GS-460V), POE lanolin (NIKKOL TW-20), and triPOE (4) lauryl ether phosphate (NIKKOL TLP-4).

Examples of the emulsifier having an HLB of 13.4 include polyglycerol monostearate (SY Glyster MSW-7S) and polyglycerol monolaurate (SY Glyster ML-500).

Examples of the emulsifier having an HLB of 13.5 include polyoxyethylene glyceryl stearate (NIKKOL TMGS-15V), POE (50) hydrogenated castor oil (NIKKOL HCO-50, NIKKOL HCO-50 (for pharmaceutical purpose), POE (10) cetyl ether (NIKKOL BC-10), POE (9) secondary alkyl ether (NIKKOL BT-9), and diPOE (10) (C12-15) alkyl ether phosphoric acid (NIKKOL DDP-10).

Examples of the emulsifier having an HLB of 14.0 include decaglyceryl monomyristate (NIKKOL Decaglyn 1-MF, NIKKOL Decaglyn 1-M), POE (60) sorbitol tetraoleate (NIKKOL GO-460V), and POE (60) hydrogenated castor oil (NIKKOL HCO-60, NIKKOL HCO-60 (for pharmaceutical purpose)).

Examples of the emulsifier having an HLB of 14.5 include hexaglyceryl monolaurate (NIKKOL Hexaglyn 1-L, NIKKOL Hexaglyn 1-LF), POE (25) phytostanol (NIKKOL BPSH-25), POE (9) lauryl ether (NIKKOL BL-9EX), POE (10) oleyl ether (NIKKOL BO-10V), POE (12) secondary alkyl ether (NIKKOL BT-12), polyglycerol fatty acid ester (cosmetic component display name: Polyglyceryl myristate-10, quasi-drug component display name: Decaglyceryl monomyristate) (NIKKOL Decaglyn 1-MVEX PN), decaglyceryl monomyristate (NIKKOL Decaglyn 1-MVEXF PN), lauromacrogol (NIKKOL BL-9EX (Japanese Pharmacopoeia grade)), and triglyceryl monocaprylate (NIKKOL Triglyn 1-KF).

Examples of the emulsifier having an HLB of 14.7 include polyglycel monolaurate (SY Glyster ML-750).

Examples of the emulsifier having an HLB of 14.9 include POE (20) sorbitan monostearate (NIKKOL TS-10V, NIKKOL TS-10MV, EMASOL S-120V).

Examples of the emulsifier having an HLB of 15.0 include decaglyceryl monostearate (NIKKOL Decaglyn 1-50SV), POE (20) sorbitan monoisostearate (NIKKOL TI-10V), POE (20) sorbitan monooleate (NIKKOL TO-10V), Polysorbate 80 (NIKKOL TO-10MV (Japanese Pharmacopoeia grade), NIKKOL TO-10F, NIKKOL SG-SOV2000F), POE lanolin (NIKKOL TW-30), POE (80) hydrogenated castor oil (NIKKOL HCO-80), polyethylene glycol monostearate (25E.O.) (NIKKOL MYS-25V), and Polysorbate 60 (NIKKOL TS-10F, NIKKOL SG-SSV2000F, EMASOL 0-120V).

Examples of the emulsifier having an HLB of 15.5 include decaglyceryl monolaurate (NIKKOL Decaglyn 1-LF, NIKKOL Decaglyn 1-L, NIKKOL Decaglyn 1-LVEXF), POE (6) sorbitol monolaurate (NIKKOL GL-1), POE (15) cetyl ether (NIKKOL BC-15), and polyoxyethylene phytosterol (20E.O.) (NIKKOL BPS-20).

Examples of the emulsifier having an HLB of 15.7 include decaglyceryl monomyristate (SY Glyster MM-750).

Examples of the emulsifier having an HLB of 16.0 include POE (15) oleyl ether (NIKKOL BO-15V).

Examples of the emulsifier having an HLB of 16.1 include polyglyceryl caprylate (SY Glyster MCA-750).

Examples of the emulsifier having an HLB of 16.5 include POE (100) hydrogenated castor oil (NIKKOL HCO-100), POE (20) behenyl ether (NIKKOL BB-20), POE (20) POP (4) cetyl ether (NIKKOL PBC-34, NIKKOL SG-C420), and polyethylene glycol distearate (NIKKOL CDS-6000P).

Examples of the emulsifier having an HLB of 16.7 include Polysorbate 20 (EMASOL L-120V).

Examples of the emulsifier having an HLB of 16.9 include monococonut oil fatty acid POE (20) sorbitan (NIKKOL TL-10).

Examples of the emulsifier having an HLB of 17.0 include decaglycerol laurate (RYOTO (trade name) polyglyester L-7D), POE (20) cetyl ether (NIKKOL BC-20), POE (20) oleyl ether (NIKKOL BO-20V), diPOE (10) sodium lauryl ether phosphate (NIKKOL DLP-10), and Polysorbate 20 (NIKKOL TL-10F, NIKKOL SG-SLV2000F).

Examples of the emulsifier having an HLB of 17.5 include polyethylene glycol monostearate (40E.0.) (NIKKOL MYS-40V, NIKKOL MYS-40MV).

Examples of the emulsifier having an HLB of 18.0 include POE (23) cetyl ether (NIKKOL BC-23), POE (20) stearyl ether (NIKKOL BS-20), POE (50) oleyl ether (NIKKOL BO-50V), POE (30) behenyl ether (NIKKOL BB-30), polyethylene glycol monostearate (45E.0.) (NIKKOL MYS-45V, NIKKOL MYS-45MV), polyethylene glycol monostearate (NIKKOL MYS-55V), polyethylene glycol monostearate (55E.0.) (NIKKOL MYS-55MV), and polyoxyethylene phytosterol (NIKKOL BPS-30).

Examples of the emulsifier having an HLB of 18.5 include POE (25) cetyl ether (NIKKOL BC-25).

Examples of the emulsifier having an HLB of 19.0 include POE (21) lauryl ether (NIKKOL BL-21) and lauromacrogol (NIKKOL BL-21 (Japanese Pharmacopoeia grade)).

Examples of the emulsifier having an HLB of 19.5 include POE (25) lauryl ether (NIKKOL BL-25), POE (30) cetyl ether (NIKKOL BC-30), and lauromacrogol (NIKKOL BL-25 (Japanese Pharmacopoeia grade)).

Examples of the emulsifier having an HLB of 20.0 include POE (40) cetyl ether (NIKKOL BC-40).

The solution preferably contains the polyhydric alcohol together with the urolithins and the emulsifier in order to solubilize the urolithins. The polyhydric alcohol is not particularly limited as long as the urolithins are solubilized in the emulsified composition, and examples thereof include glycerol, diglycerol, triglycerol, polyglycerol, propylene glycol, dipropylene glycol, 1,3-butylene glycol, ethylene glycol, polyethylene glycol, sorbitol (D-sorbitol), xylitol, maltitol, erythritol, mannitol, xylose, glucose, lactose, mannose, oligotose, dextrose liquid sugar, and sucrose. Among them, glycerol is preferable from the viewpoint of ensuring the fluidity of the composition.

In this step. a single type of the polyhydric alcohol may be used alone or two or more types thereof in any combination.

The content of the polyhydric alcohol in the solution containing the urolithins, the emulsifier, and the polyhydric alcohol is not particularly limited as long as the urolithins are solubilized in the emulsified composition, but for example, the total amount of the polyhydric alcohol is 25 parts by weight or more, 45 parts by weight or more, 49 parts by weight or more, 75 parts by weight or more, 100 parts by weight or more, 200 parts by weight or more, 250 parts by weight or more, 300 parts by weight or more, 450 parts by weight or more, 475 parts by weight or more, 500 parts by weight or more, 750 parts by weight or more, or 950 parts by weight or more with respect to 1 part by weight of the urolithins in total. On the other hand, the total amount of the polyhydric alcohol is, for example, 1950 parts by weight or less, 1000 parts by weight or less, 900 parts by weight or less, 490 parts by weight or less, 470 parts by weight or less, 400 parts by weight or less, 200 parts by weight or less, or 75 parts by weight or less. The upper limit and the lower limit may also be a consistent combination thereof. For example, the total amount of the polyhydric alcohol is from 25 parts by weight to 75 parts by weight, from 45 parts by weight to 75 parts by weight, from 49 parts by weight to 75 parts by weight, from 75 parts by weight to 200 parts by weight, from 100 parts by weight to 200 parts by weight, from 200 parts by weight to 400 parts by weight, from 250 parts by weight to 400 parts by weight, from 300 parts by weight to 400 parts by weight, from 450 parts by weight to 470 parts by weight, from 475 parts by weight to 490 parts by weight, from 500 parts by weight to 900 parts by weight, from 750 parts by weight to 1000 parts by weight, from 950 parts by weight to 1950 parts by weight, or the like.

In the emulsified composition produced by the production method according to the present embodiment, it is preferable that the urolithins be more solubilized, and from this viewpoint, it is preferable that the ratio of the emulsifier to the urolithins be larger. Thus, in the solution containing the urolithins, the emulsifier, and the polyhydric alcohol, the total amount of the emulsifier is, for example, 10 parts by weight or more with respect to 1 part by weight of the urolithins in total. The total amount of the emulsifier is, in ascending order of preference, 20 parts by weight or more, 40 parts by weight or more, 50 parts by weight or more, 75 parts by weight or more, 100 parts by weight or more, 150 parts by weight or more, 200 parts by weight or more, 250 parts by weight or more, 300 parts by weight or more, 350 parts by weight or more, 400 parts by weight or more, 450 parts by weight or more, 500 parts by weight or more, 550 parts by weight or more, or 600 parts by weight or more. The upper limit is not particularly limited, but is, for example, 1000 parts by weight or less. The upper limit and the lower limit may also be a consistent combination thereof. For example, the total amount of the emulsifier is from 10 parts by weight to 1000 parts by weight, from 20 parts by weight to 1000 parts by weight, from 40 parts by weight to 1000 parts by weight, from 50 parts by weight to 1000 parts by weight, from 75 parts by weight to 1000 parts by weight, from 100 parts by weight to 1000 parts by weight, from 150 parts by weight to 1000 parts by weight, from 200 parts by weight to 1000 parts by weight, from 250 parts by weight to 1000 parts by weight, from 300 parts by weight to 1000 parts by weight, from 350 parts by weight to 1000 parts by weight, from 400 parts by weight to 1000 parts by weight, from 450 parts by weight to 1000 parts by weight, from 500 parts by weight to 1000 parts by weight, from 550 parts by weight to 1000 parts by weight, from 600 parts by weight to 1000 parts by weight, or the like.

To solubilize the urolithins in the emulsified composition produced by the production method according to the present embodiment, the step (a) includes a step of heating the solution containing the urolithins, the emulsifier, and the polyhydric alcohol at 120° C. or higher for 1 minute or more. The heating time is the time after the temperature reaches the above-described temperature.

To further solubilize the urolithins in the emulsified composition produced by the production method according to the present embodiment, the temperature is, in ascending order of preference, 130° C. or higher, 140° C. or higher, 150° C. or higher, 160° C. or higher, 170° C. or higher, or 180° C. or higher. The upper limit is not particularly limited, but is, for example, 200° C. or lower. The upper limit and the lower limit may also be a consistent combination thereof. For example, the temperature is from 120° C. to 200° C., from 130° C. to 200° C., from 140° C. to 200° C., from 150° C. to 200° C., from 160° C. to 200° C., from 170° C. to 200° C., from 180° C. to 200° C., or the like.

The heating time is, in ascending order of preference, 1 minute or more, 3 minutes or more, 5 minutes or more. The upper limit is not particularly limited, but is, for example, 10 minutes or less. The upper limit and the lower limit may also be a consistent combination thereof. For example, the heating time is from 1 minute to 10 minutes, from 3 minutes to 10 minutes, from 5 minutes to 10 minutes, or the like.

In the heating step, the solution is preferably heated while being mixed. After heating, the temperature of the solution is preferably lowered while the solution is allowed to stand or is mixed at room temperature (about 25° C.).

Next, the step (b) will be described.

The step (b) of the production method according to the present embodiment is a step of heating a solution including urolithins, an emulsifier, and a polyhydric alcohol at 105° C. or higher for 1 minute or more, wherein the emulsifier has an HLB of 15.5 or greater, and a total amount of the emulsifier is 500 parts by weight or more with respect to 1 part by weight of the urolithins in total.

For the urolithins, the content described in the step (a) is incorporated.

As the emulsifier, an emulsifier having a hydrophilic-lipophilic balance (HLB) of 15.5 or greater may be used.

In this step. a single type of the emulsifier may be used alone or two or more types thereof in any combination.

It is preferable that the urolithins be more solubilized in the emulsified composition, and from this viewpoint, it is preferable that the HLB be larger. Thus, the HLB is, in ascending order of preference, 16.0 or greater, 16.1 or greater, 16.5 or greater, 16.7 or greater, 16.9 or greater, 17.0 or greater, 17.5 or greater, 18.0 or greater, 18.5 or greater, 19.0 or greater, or 19.5 or greater. The upper limit of the HLB is, for example, 20.0 or less. The upper limit and the lower limit may also be a consistent combination thereof. For example, the HLB is from 15.5 to 20.0, from 16.0 to 20.0, from 16.1 to 20.0, from 16.5 to 20.0, from 16.7 to 20.0, from 16.9 to 20.0, from 17.0 to 20.0, from 17.5 to 20.0, from 18.0 to 20.0, from 18.5 to 20.0, from 19.0 to 20.0, from 19.5 to 20.0, or the like.

As specific examples of the emulsifier, those having an HLB of 15.5 or greater among the emulsifiers described in the step (a) are incorporated.

For the polyhydric alcohol, the contents described in the step (a) are incorporated.

In the emulsified composition produced by the production method according to the present embodiment, the urolithins are preferably more solubilized, and from this viewpoint, the ratio of the emulsifier to the urolithins is preferably higher. Thus, in the solution containing the urolithins, the emulsifier, and the polyhydric alcohol, the total amount of the emulsifier is, for example, 100 parts by weight or more with respect to 1 part by weight of the urolithins in total. The total amount of the emulsifier is, in ascending order of preference, 200 parts by weight or more, 400 parts by weight or more, 500 parts by weight or more, 550 parts by weight or more, or 600 parts by weight or more. The upper limit is not particularly limited, but is, for example, 1000 parts by weight or less. The upper limit and the lower limit may also be a consistent combination thereof. For example, the total amount of the emulsifier is from 100 parts by weight to 1000 parts by weight, from 200 parts by weight to 1000 parts by weight, from 400 parts by weight to 1000 parts by weight, from 500 parts by weight to 1000 parts by weight, from 550 parts by weight to 1000 parts by weight, from 600 parts by weight to 1000 parts by weight, or the like.

To solubilize the urolithins in the emulsified composition produced by the production method according to the present embodiment, the step (b) includes a step of heating the solution containing the urolithins, the emulsifier, and the polyhydric alcohol at 105° C. or higher for 1 minute or more. The heating time is the time after the temperature reaches the above-described temperature.

To further solubilize the urolithins in the emulsified composition produced by the production method according to the present embodiment, the temperature is, in ascending order of preference, 110° C. or higher, 115° C. or higher, 120° C. or higher, 125° C. or higher, 130° C. or higher, 140° C. or higher, 150° C. or higher, 160° C. or higher, 170° C. or higher, or 180° C. or higher. The upper limit is not particularly limited, but is, for example, 200° C. or lower. The upper limit and the lower limit may also be a consistent combination thereof. For example, the temperature is from 105° C. to 200° C., from 110° C. to 200° C., from 115° C. to 200° C., from 120° C. to 200° C., from 125° C. to 200° C., from 130° C. to 200° C., from 140° C. to 200° C., from 150° C. to 200° C., from 160° C. to 200° C., from 170° C. to 200° C., or from 180° C. to 200° C.

The heating time is, in ascending order of preference, 1 minute or more, 3 minutes or more, 5 minutes or more. The upper limit is not particularly limited, but is, for example, 10 minutes or less. The upper limit and the lower limit may also be a consistent combination thereof. For example, the heating time is from 1 minute to 10 minutes, from 3 minutes to 10 minutes, from 5 minutes to 10 minutes, or the like.

In the heating step, the solution is preferably heated while being mixed. After heating, the temperature of the solution is preferably lowered while the solution is allowed to stand or is mixed at room temperature (about 25° C.).

In another embodiment of the present disclosure, an emulsified composition contains urolithins, an emulsifier, and a polyhydric alcohol, wherein the emulsifier has an HLB of 12.0 or greater, a total amount of the emulsifier is 50 parts by weight or more with respect to 1 part by weight of the urolithins in total, and the emulsified composition has a transmittance of 40% or greater for light having a wave length of 660 nm at a measurement optical path length of 10 mm.

The emulsified composition according to the present embodiment can be produced by the production method according to the above embodiment.

For the detailed description of the emulsified composition according to the present embodiment, including the urolithins, the emulsifier, and the polyhydric alcohol of the emulsified composition according to the present embodiment, the HLB of the emulsifier, the total amount of the emulsifier with respect to 1 part by weight of the urolithins in total, and the transmittance for light having a wave length of 660 nm at a measurement optical path length of 10 mm, the description of the above embodiment is incorporated.

For example, the emulsified composition according to the present embodiment may contain urolithins, an emulsifier, and a polyhydric alcohol, wherein the emulsifier has an HLB of 15.5 or greater, a total amount of the emulsifier is 500 parts by weight or more with respect to 1 part by weight of the urolithins in total, and the emulsified composition has a transmittance of 40% or greater for light having a wave length of 660 nm at a measurement optical path length of 10 mm.

As can be seen from the examples described below, when the emulsified composition according to the present embodiment is administered to a subject, the bioavailability of the urolithins in the subject is significantly higher than when the urolithins in the form of a crystalline powder is administered.

Examples of the subject include mammals. Examples of the mammals include humans, mice, rats, guinea pigs, hamsters, cows, goats, sheep, pigs, monkeys, dogs, and cats, without particular limitation.

The content of the urolithins as a total amount with respect to the whole amount of the emulsified composition can be appropriately set in accordance with the effect exhibited by the urolithins. The content of the urolithins as a total amount with respect to the whole amount of the emulsified composition is, for example, 0.001 wt. % or greater, 0.005 wt. % or greater, 0.01 wt. % or greater, 0.05 wt. % or greater, 0.1 wt. % or greater, 0.5 wt. % or greater, or 1.0 wt. % or greater. The total amount of the urolithins is, for example, 10 wt. % or less, 5 wt. % or less, 2.5 wt. % or less, or 2 wt. % or less. The upper limit and the lower limit may also be a consistent combination thereof. For example, the total amount of the urolithins is from 0.001 wt. % to 10 wt. %, from 0.005 wt. % to 5 wt. %, from 0.01 wt. % to 2.5 wt. %, from 0.05 wt. % to 2 wt. %, from 0.1 wt. % to 2 wt. %, from 0.5 wt. % to 2 wt. %, from 1.0 wt. % to 2 wt. %, or the like.

The intake amount (or administration amount), the intake (or administration) schedule, and the like as the total amount of the urolithins can be appropriately set according to the effect exerted by the urolithins.

The intake amount (or administration amount) as the total amount of urolithins is, for example, 0.05 mg or more, 0.1 mg or more, or 0.15 mg or more, and on the other hand, 40 mg or less, 20 mg or less, or 10 mg or less per day and kg body weight. For example, the intake amount is from 0.05 mg to 40 mg, from 0.1 mg to 20 mg, or from 0.15 mg to 10 mg.

Another embodiment of the present disclosure is a product containing the emulsified composition according to the above embodiment.

Examples of the product include food and beverage products, pharmaceutical products, and cosmetic products.

When the product is a food or beverage product, the food or beverage product may be composed of the emulsified composition according to the above embodiment, or may be a food or beverage product containing other raw materials. The food or beverage product includes a supplement.

When the emulsified composition according to the present embodiment is used as a material for food or beverage products or a food or beverage product, in addition to general food and beverage products, the food or beverage product may be a food for specified health uses, a dietary supplement, a functional food, a food for a sick person, and a food additive (beverages are also included in these).

The food or beverage product may take a form suitable for eating, such as a granular form, a particle form, a tablet form, a capsule form, or a paste form, for example, by adding an appropriate auxiliary agent thereto and using a known method.

The emulsified composition according to the embodiment may be added to various food and beverage products, for example, processed meat foods such as hams and sausages, processed marine foods such as fish sausages, breads, confectioneries, butters, powdered milk, fermented milk products, and beverages such as juices, milks, and cold beverages.

The food or beverage product can contain, as a main ingredient, water, a protein, a carbohydrate, a lipid, vitamins, minerals, an organic acid, an organic base, a fruit juice, a flavor, or the like. Examples of the protein include animal and vegetable proteins, such as whole milk powders, skimmed milk powders, partially skimmed milk powders, casein, soy proteins, chicken egg proteins, and meat proteins; their hydrolysates; and butters. Examples of the carbohydrate include sugars, processed starches (dextrin, as well as soluble starches, British starch, oxidized starches, starch esters, starch ethers, and the like), and dietary fibers. Examples of the lipid include lard; and vegetable oils and fats, such as safflower oil, corn oil, rapeseed oil, coconut oil, and their fractionated oils, hydrogenated oils, and transesterified oils. Examples of the vitamins include vitamin A, carotenes, B-group vitamins, vitamin C, D-group vitamins, vitamin E, K-group vitamins, vitamin P, vitamin Q, niacin, nicotinic acid, pantothenic acid, biotin, inositol, choline, and folic acid, and examples of the mineral include calcium, potassium, magnesium, sodium, copper, iron, manganese, zinc, selenium, and whey minerals. Examples of the organic acid include malic acid, citric acid, lactic acid, and tartaric acid. Two or more types of these components may be used in combination. Synthetic products of these components and/or food or beverage products containing these components in large amounts may be used.

The food or beverage product can be produced, for example, by a common method. The blending amount of the emulsified composition into a food or beverage product, the blending method, and the blending time can be selected as appropriate. That is, the food or beverage product may be produced through a step of producing an emulsified composition by the production method according to the above embodiment, and then blending the emulsified composition and a raw material of the food or beverage product to produce the food or beverage product. The food or beverage product can be sealed in an appropriate container, such as a bottle, a bag, a can, a box, or a pack, as necessary.

The content as the total amount of urolithins with respect to the whole amount of the food or beverage product, the intake amount as the total amount of urolithins, the intake schedule, and the like can be appropriately set in accordance with the effects exhibited by the urolithins. For example, as the content and the intake amount, the content (already described) as the total amount of the urolithins with respect to the whole amount of the emulsified composition and the intake amount (already described) as the total amount of the urolithins can be incorporated.

When the product is a pharmaceutical product, the pharmaceutical product may be composed of the emulsified composition according to the embodiment, or may be a pharmaceutical product containing other raw materials.

When the emulsified composition is used as a material for a pharmaceutical product or a pharmaceutical product, the method for application of the pharmaceutical product that can be employed is either oral administration or parenteral administration. When the pharmaceutical product is administered, the active ingredient is mixed with a solid or liquid non-toxic pharmaceutical carrier suitable for an administration method, such as oral administration, intrarectal administration, or injection, and then can be administered in a commonly used form of a pharmaceutical formulation.

Examples of such a formulation include solid agents, such as tablets, granules, powders, and capsules; liquid agents, such as solutions, suspensions, and emulsions; and lyophilized formulations. These formulations can be prepared by common means for formulation preparation. Examples of the non-toxic pharmaceutical carrier described above include glucose, lactose, sucrose, starch, mannitol, dextrin, fatty acid glyceride, poly(ethylene glycol), hydroxyethyl starch, ethylene glycol, polyoxyethylene sorbitan fatty acid esters, amino acids, gelatin, albumin, water, and physiological saline. In addition, as necessary, a commonly used additive, such as a stabilizer, a wetting agent, an emulsifier, a binder, or an isotonizing agent, can be appropriately added.

The pharmaceutical product can be produced, for example, by a common method. The blending amount of the emulsified composition into a pharmaceutical product, the blending method, and the blending time can be selected as appropriate. That is, after the emulsified composition is produced by the production method according to the above embodiment, the pharmaceutical product may be produced through a step of blending the emulsified composition and a raw material of the pharmaceutical product to produce the pharmaceutical product. The pharmaceutical product can be sealed in an appropriate container, such as a bottle, a bag, a can, a box, or a pack, as necessary.

The content as the total amount of the urolithins with respect to the whole amount of the pharmaceutical product, the administration amount as the total amount of the urolithins, the administration schedule, and the like can be appropriately set in accordance with the effects exhibited by the urolithins. For example, as the content and the administration amount, the content (already described) as the total amount of the urolithins with respect to the whole amount of the emulsified composition and the administration amount (already described) as the total amount of the urolithins can be incorporated.

The pharmaceutical product can be used for preventing or ameliorating a disease (including symptoms, signs, disorders) that can be prevented or ameliorated by administration of the urolithins. Examples of the diseases include dementia caused from AIDS, amyotrophic lateral sclerosis, adrenal leukodystrophy, Alexander's disease, Alpers' disease, ataxia telangiectasia, Batten's disease, bovine spongiform encephalopathy (BSE), Kanaban's disease, corticobiliary degeneration, Creutzfeld-Jakob disease, dementia with Lewy bodies, fatal familial insomnia, frontotemporal lobar degeneration, Huntington's disease, Kennedy's disease, Krabbe's disease, Lyme disease, Machado-Joseph disease, multiple sclerosis, multiple system atrophy, neuroacanthocytosis, Niemann's disease, Parkinson's disease, Pick's disease, spinocerebellar ataxia, and subacute spinal cord associated degeneration described in JP 6871304 B; mood disorders, atherosclerosis, macular degeneration, sensory deafness, infection diseases, myocardial infarction, cardiac bypass, dermatitis, psoriasis, cardiac hypertrophy, left ventricular enlargement, reduced cardiac output, reduced cardiac function, Danon disease, muscle inactivation atrophy, skeletal muscle atrophy, cirrhosis of the liver, autoimmune disease, lupus erythematosus, Alzheimer's disease, hypertension, chronic kidney disease, sickle cell anemia, chronic obstructive pulmonary disorder, acute pancreatitis, heart disease, endophthalmitis, uveitis, emphysema, human idiopathic pulmonary fibrosis, immune response to infection by pathogens, and diabetic retinopathy described in JP 6879980 B; obesity, reduced metabolic rate, metabolic syndrome, diabetes, and hyperlipidemia described in JP 6254667 B.

“Amelioration” includes treatment.

When the product is a cosmetic product, the cosmetic product may be composed of the emulsified composition according to the above embodiment, or may be a cosmetic product containing other raw materials.

When the emulsified composition according to the above embodiment is used as a material of a cosmetic product or a cosmetic product, examples of the form of the cosmetic product include various desired forms such as a liquid form including an aqueous solution, a lotion, a spray liquid, a suspension, and an emulsion; a solid form including a powder, a granule, and a block; a semi-solid form including a cream and a paste; and a gel.

Examples of the cosmetic product include facial cleansing cosmetics, skin cosmetics (e.g., lotion, emulsion, cream, etc.), sunscreen cosmetics, makeup cosmetics (e.g., foundation, lipstick, etc.), cleansing cosmetics (e.g., facial cleansing foam, solid soap, body shampoo, shampoo, conditioner, rinse, etc.), and scalp cosmetics (e.g., hair tonic, hair liquid, hair-raising agent, hair growth, etc.).

The cosmetic product can be produced by appropriately adding components used in general cosmetic products or components necessary for formulation. Examples of such components include water-soluble polymers, plant components, humectants, waxes, hydrocarbons, essential oils, oil and fat components (emollient components), inorganic salts, pigments, fragrances, fine powders, bactericides, and preservatives.

The cosmetic product can be produced, for example, by a common method. The blending amount of the emulsified composition into a cosmetic product, the blending method, and the blending time can be selected as appropriate. That is, after the emulsified composition is produced by the production method according to the above embodiment, the cosmetic product may be produced through a step of blending the emulsified composition and a raw material of the cosmetic product to form the cosmetic product. The cosmetic product can be sealed in an appropriate container, such as a bottle, a bag, a can, a box, or a pack, as necessary.

The content of the urolithins as a total amount with respect to the whole amount of the cosmetic product, the use amount of the urolithins as a total amount, the schedule of use, and the like can be appropriately set in accordance with the effects exhibited by the urolithins. For example, as the content and the use amount, the content (already described) as the total amount of the urolithins with respect to the whole amount of the emulsified composition and the intake amount (already described) as the total amount of the urolithins can be incorporated.

As described above, when the emulsified composition according to the embodiment is administered to a subject, the bioavailability of the urolithins in the subject is significantly higher than when the urolithins in the form of a crystalline powder is administered.

Thus, the present disclosure can provide the following as an embodiment.

A method including steps of:
(I): producing an emulsified composition having a transmittance of 40% or greater for light having a wavelength of 660 nm at a measurement optical path length of 10 mm; and
(II): administering an effective amount of the emulsified composition produced in (I) to a subject in need of administration of urolithins,
wherein the step (I) includes the following steps (a) or (b):
(a)
heating a solution containing urolithins, an emulsifier, and a polyhydric alcohol at 120° C. or higher for 1 minute or more, wherein the emulsifier has an HLB of 12.0 or greater, and a total amount of the emulsifier is 50 parts by weight or more with respect to 1 part by weight of the urolithins in total.
(b)
heating a solution containing urolithins, an emulsifier, and a polyhydric alcohol at 105° C. or higher for 1 minute or more, wherein the emulsifier has an HLB of 15.5 or greater, and a total amount of the emulsifier is 500 parts by weight or more with respect to 1 part by weight of the urolithins in total.

As the effective amount, the administration amount (described above) as the total amount of urolithins can be incorporated. That is, for example, the effective amount is 0.05 mg or more, 0.1 mg or more, or 0.15 mg or more, and on the other hand, 40 mg or less, 20 mg or less, or 10 mg or less per day and kg body weight. For example, the effective amount is from 0.05 mg to 40 mg, from 0.1 mg to 20 mg, or from 0.15 mg to 10 mg.

The present disclosure can also provide the following as an embodiment.

A method including administering an effective amount of an emulsified composition to a subject in need of administration of urolithins,
wherein
the emulsified composition contains urolithins, an emulsifier, and a polyhydric alcohol,
the emulsifier has an HLB of 12.0 or greater,
a total amount of the emulsifier is 50 parts by weight or more with respect to 1 part by weight of the urolithins in total, and
the emulsified composition has a transmittance of 40% or greater for light having a wavelength of 660 nm at a measurement optical path length of 10 mm.

As the effective amount, the administration amount (described above) as the total amount of urolithins can be incorporated. That is, for example, the effective amount is 0.05 mg or more, 0.1 mg or more, or 0.15 mg or more, and on the other hand, 40 mg or less, 20 mg or less, or 10 mg or less per day and kg body weight. For example, the effective amount is from 0.05 mg to 40 mg, from 0.1 mg to 20 mg, or from 0.15 mg to 10 mg.

### EXAMPLES

Examples will be described below, but none of the examples shall be construed as limiting the present disclosure.

### (Method for Measuring Transmittance)

The transmittance of the emulsified composition produced in this example was determined as a transmittance for light having a wave length of 660 nm at a measurement optical path length of 10 mm using an ultraviolet-visible spectrophotometer UV-1800 (manufactured by Shimadzu Corporation). In the following test, a case where the transmittance was less than 40% was indicated by “C”, a case where the transmittance was 40% or greater and less than 80% was indicated by “B”, and a case where the transmittance was 80% or greater was indicated by “A”.

### Test Example 1

A solution having a composition of parts by weight shown in Table 1 (a solution in which the balance is glycerol and the total is 100 parts by weight) was prepared and heated at a heating temperature shown in Table 1 for 3 minutes using a block heater. During being heated, the solution was mixed using a polytron homogenizer at 10000 rpm. Thereafter, the solution was allowed to stand at room temperature (about 25° C.) and the temperature of the solution was lowered to room temperature (about 25° C.) to produce an emulsified composition, and the transmittance was measured.

The results are shown in Table 1.

TABLE 1

| | HLB | Parts by weight | | | | | | | | | | |
|---|---|---|---|---|---|---|---|---|---|---|---|---|
| Urolithin A | — | 0.1 | 0.1 | 0.1 | 0.1 | 0.1 | 0.1 | 0.1 | 0.1 | 0.1 | 0.1 | 0.1 |
| RYOTO | 7 | 5 | 5 | 5 | | | | | | | | |
| polyglyester O-50D | | | | | | | | | | | | |
| Sugar ester S-1170 | about | | | | 5 | 5 | 5 | | | | | |
| | 11 | | | | | | | | | | | |
| Decaglyn 1-OVF | 12.0 | | | | | | | 5 | 5 | 5 | | |
| RYOTO | 13.0 | | | | | | | | | | 5 | 5 |
| polyglyester O-15D | | | | | | | | | | | | |
| Decaglyn 1-MF | 14.0 | | | | | | | | | | | |
| Decaglyn 1-LF | 15.5 | | | | | | | | | | | |
| RYOTO | 17.0 | | | | | | | | | | | |
| polyglyester L-7D | | | | | | | | | | | | |
| Heating | | 80 | 100 | 120 | 80 | 100 | 120 | 80 | 100 | 120 | 80 | 100 |
| temperature (° C.) | | | | | | | | | | | | |
| Transmittance | | C | C | C | C | C | C | C | C | B | C | C |
| results | | | | | | | | | | | | |

| | Parts by weight | | | | | | | | | |
|---|---|---|---|---|---|---|---|---|---|---|
| Urolithin A | 0.1 | 0.1 | 0.1 | 0.1 | 0.1 | 0.1 | 0.1 | 0.1 | 0.1 | 0.1 |
| RYOTO | | | | | | | | | | |
| polyglyester O-50D | | | | | | | | | | |
| Sugar ester S-1170 | | | | | | | | | | |
| Decaglyn 1-OVF | | | | | | | | | | |
| RYOTO | 5 | | | | | | | | | |
| polyglyester O-15D | | | | | | | | | | |
| Decaglyn 1-MF | | 5 | 5 | 5 | | | | | | |
| Decaglyn 1-LF | | | | | 5 | 5 | 5 | | | |
| RYOTO | | | | | | | | 5 | 5 | 5 |
| polyglyester L-7D | | | | | | | | | | |
| Heating | 120 | 80 | 100 | 120 | 80 | 100 | 120 | 80 | 100 | 120 |
| temperature (° C.) | | | | | | | | | | |
| Transmittance | B | C | C | B | C | C | B | C | C | A |
| results | | | | | | | | | | |

### Test Example 2

A solution having a composition of parts by weight shown in Table 2 (a solution in which the balance is glycerol and the total is 100 parts by weight) was prepared and heated at 120° C. for 3 minutes using a block heater. During being heated, the solution was mixed using a polytron homogenizer at 15000 rpm. Thereafter, the solution was allowed to stand at room temperature (about 25° C.) and the temperature of the solution was lowered to room temperature (about 25° C.) to produce an emulsified composition, and the transmittance was measured.

The results are presented in Table 2.

TABLE 2

| | HLB | Parts by weight | | | | | | |
|---|---|---|---|---|---|---|---|---|
| Urolithin A | — | 0.2 | 0.2 | 0.2 | 0.2 | 0.2 | 0.2 | 0.5 |
| Decaglyn 1-LF | 15.5 | 5 | 10 | 50 | | | | |
| RYOTO | 17.0 | | | | 5 | 10 | 50 | 50 |
| polyglyester L-7D | | | | | | | | |
| Heating | | 120 | | | 120 | | | |
| temperature (° C.) | | | | | | | | |
| Transmittance | | C | B | A | C | B | A | A |
| results | | | | | | | | |

### Test Example 3

A solution having a composition of parts by weight shown in Table 3 (a solution in which the balance is glycerol and the total is 100 parts by weight) was prepared and heated at a heating temperature shown in Table 3 for 3 minutes using a block heater. During being heated, the solution was mixed using a polytron homogenizer at 15000 rpm. Thereafter, the solution was allowed to stand at room temperature (about 25° C.) and the temperature of the solution was lowered to room temperature (about 25° C.) to produce an emulsified composition, and the transmittance was measured.

The results are presented in Table 3.

TABLE 3

| | HLB | Parts by weight | | | | | | | | | | | |
|---|---|---|---|---|---|---|---|---|---|---|---|---|---|
| Urolithin A | — | 0.1 | 0.1 | 0.1 | 0.1 | 0.1 | 0.1 | 0.1 | 0.1 | 0.1 | 0.1 | 0.1 | 0.1 |
| Decaglyn 1-LF | 15.5 | 5 | 5 | 5 | 50 | 50 | 50 | | | | | | |
| RYOTO | 17.0 | | | | | | | 5 | 5 | 5 | 50 | 50 | 50 |
| polyglyester L-7D | | | | | | | | | | | | | |
| Heating | | 100 | 105 | 120 | 100 | 105 | 120 | 100 | 105 | 120 | 100 | 105 | 120 |
| temperature (° C.) | | | | | | | | | | | | | |
| Transmittance | | C | C | B | C | A | A | C | C | A | C | A | A |
| results | | | | | | | | | | | | | |

### Test Example 4

A solution having a composition of parts by weight shown in Table 4 (a solution in which the balance is glycerol and the total is 100 parts by weight) was prepared and heated at 120° C. for 3 minutes using a block heater. During being heated, the solution was mixed using a polytron homogenizer at 15000 rpm. Thereafter, the solution was allowed to stand at room temperature (about 25° C.) and the temperature of the solution was lowered to room temperature (about 25° C.) to produce an emulsified composition, and the transmittance was measured.

The results are presented in Table 4.

TABLE 4

| | HLB | Parts by weight | | | | | | |
|---|---|---|---|---|---|---|---|---|
| Urolithin B | — | 1.0 | | | | | | |
| Urolithin C | — | | 0.1 | | | | | |
| Isourolithin A | — | | | 0.5 | | | | |
| Urolithin M5 | — | | | | 0.1 | | | |
| Urolithin M6 | — | | | | | 0.5 | | |
| Urolithin M7 | — | | | | | | 0.1 | |
| Urolithin M8 | — | | | | | | | 0.5 |
| RYOTO | 17.0 | 50 | 50 | 50 | 50 | 50 | 50 | 50 |
| polyglyester | | | | | | | | |
| L-7D | | | | | | | | |
| Heating | | 120 | | | | | | |
| temperature | | | | | | | | |
| (° C.) | | | | | | | | |
| Transmittance | | A | A | B | A | A | A | A |
| results | | | | | | | | |

### Test Example 5

A solution having a composition of parts by weight shown in Table 5 (a solution in which the balance is glycerol and the total is 100 parts by weight) was prepared and heated at 120° C. for 3 minutes using a block heater. During being heated, the solution was mixed using a polytron homogenizer at 15000 rpm. Thereafter, the solution was allowed to stand at room temperature (about 25° C.) and the temperature of the solution was lowered to room temperature (about 25° C.) to produce an emulsified composition, which was used in the test group described below. The conditions are the same as those of one example in Table 2, and it is apparent from Table 2 that the transmittance of the emulsified composition is “A”.

TABLE 5

| | HLB | Parts by weight |
|---|---|---|
| Urolithin A | — | 0.5 |
| RYOTO polyglyester L-7D | 17.0 | 50 |

Six 5-week-old male SD rats were preliminarily reared for one week, fasted for 24 hours, and divided into two groups (test group and control group).

### (Test Group)

Carboxymethyl cellulose (Kanto Scientific Co., Ltd.) was dissolved in Japanese Pharmacopeia water for injection (Otsuka Pharmaceutical Co., Ltd.) to have a final concentration of 0.5%, and the emulsified composition was mixed therewith to have a urolithin A concentration of 10 mg/kg BW, and the mixture was administered to the rats of the test group.

Blood was collected at a total of 8 time points, pre-dose on the day of dosing, 0.5, 1, 2, 4, 8, 12, and 24 hours post-dose. Blood was collected using isoflurane (Isoflu, Zoetis Japan Ltd.) under light anesthesia, the injection was performed through a jugular vein using a syringe and a needle (both TERUMO CORPORATION) treated with sodium heparin (heparin Na 5000 units/5 mL for injection, “MOCHIDA”, MOCHIDA PHARMACEUTICAL CO., LTD.). Blood was collected from the abdominal aorta 24 hours after the administration as collection of whole blood. The collected blood was transferred to a blood collection tube and centrifuged at 4° C. and 3500 rpm for 10 minutes to separate plasma.

A PBS buffer solution in an amount of 4 times of the plasma per sample was added and mixed. Thereafter, to 250 μL of this solution, 50 μL of 50 mM ascorbic acid was added, and 40 units of sulfatase (Sigma-Aldrich) was further added. After being sufficiently mixed, the mixture was heated at 37° C. for 2 hours. Further, 350 μL of ethyl acetate was added and the mixture was mixed well, and then subjected to centrifugation at 3000 rpm for 1 minute to collect the ethyl acetate layer. This operation was repeated three times, the resulting material was concentrated and dried, and 50 μL of methanol was added to the concentrated and dried material to prepare a sample for analysis.

The amount of urolithin A in the sample for analysis was determined by HPLC under the following conditions. A solution prepared by dissolving urolithin A (Cayman Chemical) in dimethyl sulfoxide (DMSO) was analyzed, and the factor of urolithin A and the concentration of urolithin A in the sample were calculated by the following calculation formulas (1) and (2) using the purity (%) (A) and the peak area value (B) in HPLC.

$$( Calculation Formula for Factor of Urolithin A )$$

$$Factor of urolithin A = ( B ) / ( concentration of urolithin A standard solution ( mg / L ) \times ( A ) / 100 ) \qquad ( 1 )$$

$$( Calculation Formula for Urolithin A Concentration of Sample )$$

$$Urolithin A concentration of sample ( mg / L ) = peak area value of urolithin A in sample / urolithin A factor \qquad ( 2 )$$

### (HPLC Analysis Conditions)

Analytical column: Inertsil ODS-3 (250×4.6 mm) (GL Science)
Detection wavelength: 305 nm
Mobile phase: water/acetonitrile/acetic acid=74/25/1
Column temperature: 40° C.
Flow rate: 1.0 mL/min

Under the above conditions, urolithin A had a retention time of 16.5 min.

An approximate value of an area under the blood concentration-time curve (AUC), which is a useful index for evaluating bioavailability of a drug or the like, was calculated from the analysis result by HPLC.

### (Control Group)

The same steps as the steps described for the above test group were performed except for the following.

Carboxymethyl cellulose (Kanto Scientific Co., Ltd.) was dissolved in Japanese Pharmacopeia water for injection (Otsuka Pharmaceutical Factory Co., Ltd.) to have a final concentration of 0.5%, and the resulting solution was mixed with crystalline powder urolithin A (Cayman Chemical Co., Ltd.) to have a urolithin A concentration of 10 mg/kg BW, and the mixture was administered to the rats of the control group.

### (Results)

The results are presented in Table 6. The AUC of the test group was about 1.5 times the AUC of the control group. That is, it was confirmed that when the emulsified composition was administered to a subject, the bioavailability of the urolithins in the subject was significantly higher than when the urolithins in the form of a crystalline powder was administered.

TABLE 6

| | Urolithin A administration | Cmax | AUC |
|---|---|---|---|
| | amount (mg/kg · BW) | (ng/mL) | (ng/mL · h) |
| Test group | 10 | 2,963 | 21,404 |
| Control group | 10 | 1,547 | 14,438 |

1. A method for producing an emulsified composition, the method comprising steps (a) or (b),
wherein the emulsified composition has a transmittance of 40% or greater for light having a wavelength of 660 nm at a measurement optical path length of 10 mm:
(a)
heating a solution containing urolithins, an emulsifier, and a polyhydric alcohol at 120° C. or higher for 1 minute or more, wherein the emulsifier has an HLB of 12.0 or greater, and a total amount of the emulsifier is 50 parts by weight or more with respect to 1 part by weight of the urolithins in total;
(b)
heating a solution containing urolithins, an emulsifier, and a polyhydric alcohol at 105° C. or higher for 1 minute or more, wherein the emulsifier has an HLB of 15.5 or greater, and a total amount of the emulsifier is 500 parts by weight or more with respect to 1 part by weight of the urolithins in total.

2. The method according to claim 1, wherein the polyhydric alcohol is glycerol.

3. The method according to claim 1, wherein the urolithins are urolithin A, urolithin B, urolithin C, urolithin M5, urolithin M6, urolithin M7, urolithin M8, or isourolithin A.

4-8. (canceled)

9. The method according to claim 2, wherein the urolithins are urolithin A, urolithin B, urolithin C, urolithin M5, urolithin M6, urolithin M7, urolithin M8, or isourolithin A.

10. An emulsified composition comprising urolithins, an emulsifier, and a polyhydric alcohol,
wherein
the emulsifier has an HLB of 12.0 or greater,
a total amount of the emulsifier is 50 parts by weight or more with respect to 1 part by weight of the urolithins in total, and
the emulsified composition has a transmittance of 40% or greater for light having a wave length of 660 nm at a measurement optical path length of 10 mm.

11. The emulsified composition according to claim 10, wherein the polyhydric alcohol is glycerol.

12. The emulsified composition according to claim 10, wherein the urolithins are urolithin A, urolithin B, urolithin C, urolithin M5, urolithin M6, urolithin M7, urolithin M8, or isourolithin A.

13. The emulsified composition according to claim 11, wherein the urolithins are urolithin A, urolithin B, urolithin C, urolithin M5, urolithin M6, urolithin M7, urolithin M8, or isourolithin A.

14. A product comprising the emulsified composition described in claim 10.

15. A product comprising the emulsified composition described in claim 11.

16. The product according to claim 14, wherein the product is a food or beverage product, a pharmaceutical product, or a cosmetic product.
